(19) United States
(12) Patent Application Publication
(10) Pub. No.: US 2014/0188097 A1
(43) Pub. Date: Jul. 3, 2014

(54) Method and Apparatus for Dielectric Barrier Discharge Wand Cold Plasma Device

(71) Applicant: Cold Plasma Medical Technologies, Inc., Scottsdale, AZ (US)

(72) Inventors: Gregory A. WATSON, Sanford, FL (US); Marc C. JACOFSKY, Phoenix, AZ (US); Steven A. MYERS, Scottsdale, AZ (US)

(73) Assignee: Cold Plasma Medical Technologies, Inc., Scottsdale, AZ (US)

(21) Appl. No.: 14/145,898

(22) Filed: Dec. 31, 2013

Related U.S. Application Data
(60) Provisional application No. 61/747,828, filed on Dec. 31, 2012.

(51) Int. Cl. A61B 18/04

(52) U.S. Cl. CPC A61B 18/042
USPC 606/18; 606/13

(57) ABSTRACT

A cold plasma device having a broad surface of plasma generation allowing for the efficient treatment of larger areas with the benefit of being durable, portable and able to treat almost any anatomical structure. The cold plasma device has a constant radius surface, which creates a tangential surface with an infinite number of distances between the surface edge of the substrate under treatment and the device.

### CROSS REFERENCE TO RELATED APPLICATIONS

This application claims the benefit of U.S. Provisional Application No. 61/747,828, filed Dec. 31, 2012 and entitled “Method and Apparatus for Dielectric Barrier Discharge Wand Cold Plasma Device,” which is incorporated herein by reference in its entirety.

This application is related to U.S. Provisional Application No. 60/913,369, filed Apr. 23, 2007; U.S. patent application Ser. No. 12/038,159, filed Feb. 27, 2008 (which issued as U.S. Pat. No. 7,633,231); U.S. patent application Ser. No. 13/620,118, filed Sep. 14, 2012, and U.S. patent application Ser. No. 14/103,540, filed Dec. 11, 2013, each of which are herein incorporated by reference in their entireties.

### BACKGROUND

1. Field of the Art

The present invention relates to devices and methods for cold plasma generation, and, more particularly, to such devices that are formed in the shape of a wand and methods for using same.

2. Background Art

At present, non-thermal (i.e., cold) atmospheric pressure plasmas for the treatment of biological substrates are found in two generalized forms. One form is the gas jet plasma, as exemplified by U.S. Provisional Application No. 60/913,369 and related matters (“the '369 family) and KinPen (PCT/EP2010/061166 application and related matters), which provide a jet of ions and reactive species that can be directed to a target over varying distances, specifically distances greater than a few millimeter. A second form is the Floating Electrode Dielectric Barrier Discharge (FE-DBD) devices, as known from the work of Fridman (PCT/US2010/027411 application), in which the target substrate (often the human body) acts as a floating ground electrode. By acting as the floating ground, the target directly attracts the electrical energy built up on the electrode until an arc, or plurality of arcs, is initiated. This arc generates ions in the atmosphere and drives those ions and reactive species to the target substrate. However, all conventional non-thermal DBD devices that utilize this floating electrode strategy are limited by the size of the area they can treat and the limited amount of variance allowed to the target treatment distance, generally less than 2 mm distance. When attempts are made to increase the size of the electrode, and thereby the surface area of treatment, or increase the distance from the target that an arc can be initiated, the heat generated by the electrode increases and can cause thermal destruction of sensitive substrates such as skin. These disadvantages are discussed further below.

Based on their electrode sizes required for non-thermal plasma output, typical FE-DBD devices can treat only a limited area at carefully controlled distances. FIGS. 1, 2, and 3 illustrate the relative small size of the plasma produced by these typical FE-DBD devices and the methods for directly controlling electrode-target distances. All common single frequency cold plasma power supplies are limited in the amount of energy they can deliver to a target before thermal effects are initiated. As a result, all electrodes connected to these power supplies are limited in their relative design size and consequently the surface area that they can effectively treat. Clearly, maintaining the optimal target distance is a critical parameter that must be precisely maintained when operating conventional FE-DBD plasma devices. When placed too close to the treatment target (<1 mm), the desired reactive species and ions are not adequately delivered to the substrate and when placed too far, no plasma is initiated. Based on this requirement, numerous methods, all using ancillary devices, have been attempted in the prior art.

As mentioned above, the distance to the treatment area for FE-DBD devices must be carefully controlled for safety and to achieve a desired effect. FIG. 1 (adapted from Fridman et al., “Use of Non-Thermal Atmospheric Pressure Plasma Discharge for Coagulation and Sterilization of Surface Wounds,” 2005) shows the use of a positioner 110 for positioning DBD device (high voltage port 130, teflon coating 120, copper electrode 140 inside quartz dielectric 150) for application to blood sample 160 in holder 170 that is in contact with ground 180. The positioner allows the entire electrode construct to be moved vertically in small increments to achieve the desired treatment distance. Existing DBD devices of this type have a flat planar structure (FIG. 2) and therefore maintaining an accurate degree of distance control is a significant challenge. An additional confounding factor to controlling the distance between these devices and the wound is the potentially varying wound bed that also alters the distance between the plasma-generating device and the patient. This means that certain portions of the wound bed will be closer to the DBD device while other portions are farther from the device. Given the variation in treatment distance, a fixed positioner's value is marginal in real-world applications.

Other prior art cold plasma devices employ ring adapters (see, for example, FIG. 3 (adapted from Dobrynin et al, “Live Pig Skin Tissue and Wound Toxicity of Cold Plasma Treatment,” 2011) where the ring adapter is represented by the 1.5 mm spacer) of various heights. These ring shaped stand-offs come into direct contact with the surface and are used to control the distance between the treatment device and the treatment substrate. However, they cannot adjust to complex wound bed architecture and cannot be moved across a surface to facilitate larger treatment areas. It is important to note that the prior art cold plasma devices illustrated in FIGS. 1, 2 and 3 are not cylindrical devices, but are planar devices.

Referring to FIG. 4 (adapted from Fridman et al., “Use of Non-Thermal Atmospheric Pressure Plasma Discharge for Coagulation and Sterilization of Surface Wounds,” 2005), a small diameter (3 mm) cylindrical electrode is depicted that maintains an optimal treatment distance by the relative diameter of the “wheels” on the carrier for the treatment device. This design can follow the general contours of the treatment surface, however the wheels could potentially contact open wound areas. In addition, this type of DBD device does not allow for the continuous micro-adjustments that would be necessary during an actual treatment session on a living patient having a complex biological and wound architecture.

What is needed is a DBD electrode design that can be easily manipulated over a plurality of surfaces while maintaining an ideal treatment distance as an inherent property of the electrode construction.

### BRIEF SUMMARY OF THE INVENTION

An embodiment is described of a cold plasma dielectric barrier discharge (DBD) device that is coupled to a power supply. The cold plasma DBD device has a wand-like shape. The wand-like shape can include a radius tip at the end of the wand-like device, and is round in cross-section, creating a tangential surface with a large number of distances between a relatively flat surface of a target substrate under treatment and the cold plasma DBD device. The length and diameter of the device can vary greatly depending on the desired size of the surface to be treated. Lengths of up to 1 meter with diameters of up to 40 mm have been constructed and successfully generate non-thermal plasma that is effective in surface modification and pathogen destruction. This longer, larger diameter device configuration effectively treats a much larger area and also allow for a greater variance in the target distance. This is achieved by creating a larger tangential surface treatment area, which helps maintain the optimal ˜2 mm or less target distance through the radius of curvature inherent to the wand design. This benefit also translates into the more effective treatment of complex biological and wound architecture based on the resulting optimal plasma distance exposure. In addition, no part of the device that is not generating plasma comes into direct contact with the treatment surface. This helps minimize potential contamination or surface irritation. Wands of this size are not generally possible with single frequency high voltage power supplies but are effectively powered by multi-frequency harmonic-rich power supplies as disclosed in the '369 family (see paragraph [0026] below).

A further embodiment is described of a method of producing cold plasma. The method includes receiving, from a power supply, electrical energy at a cold plasma dielectric barrier discharge (DBD) device. The cold plasma DBD device has a wand-like shape. The wand-like shape can include a radius tip at the end of the wand-like device, is round in cross-section, creating a large number of distances between a relatively flat surface of a target substrate under treatment and the cold plasma DBD device. The method also includes outputting the cold plasma at the target substrate over an effective area.

### BRIEF DESCRIPTION OF THE DRAWINGS/FIGURES

FIG. 1 illustrates a floating electrode DBD device utilizing a Z-Micro positioner.

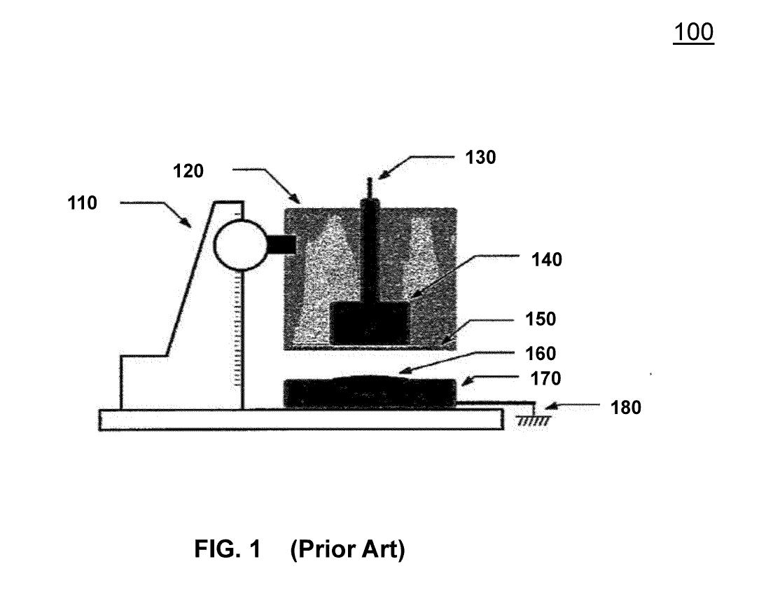

FIG. 2 illustrates the functioning surface of a floating electrode DBD device.

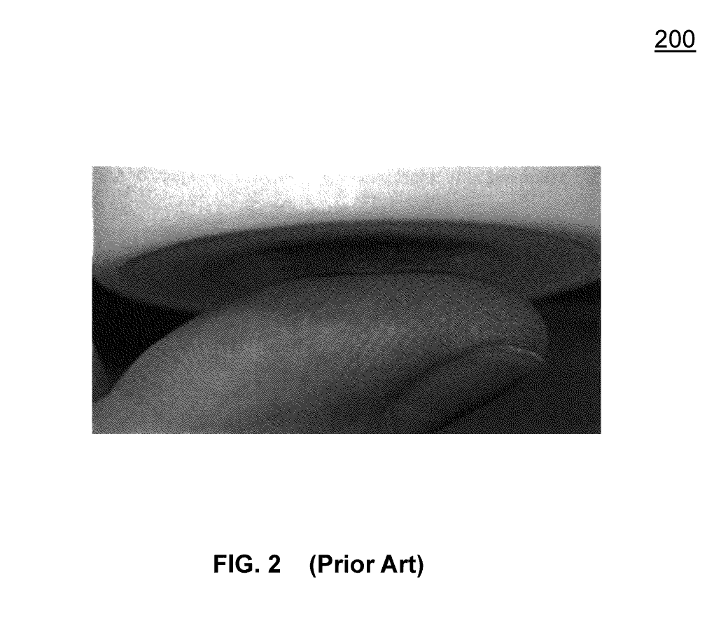

FIGS. 3A and 3B illustrate floating electrode DBD devices configured into modified planar designs.

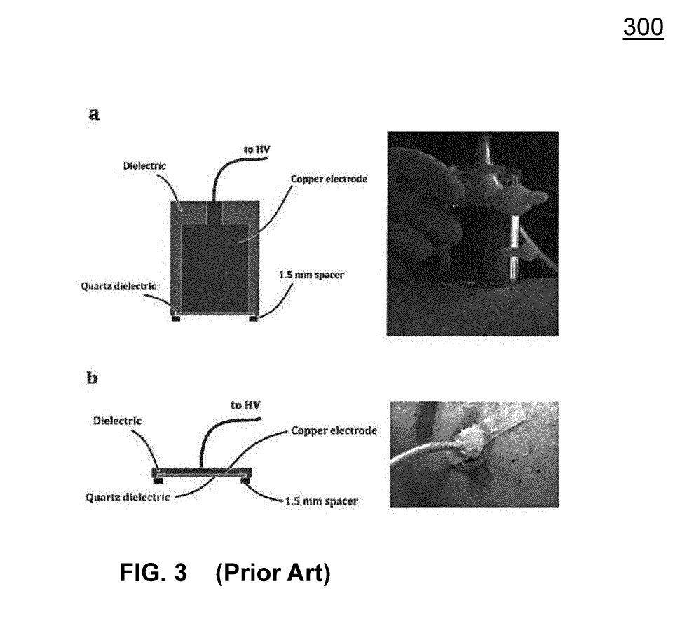

FIG. 4 illustrates a schematic view of a floating electrode DBD cylindrical device and the same device incorporated into a wheeled housing.

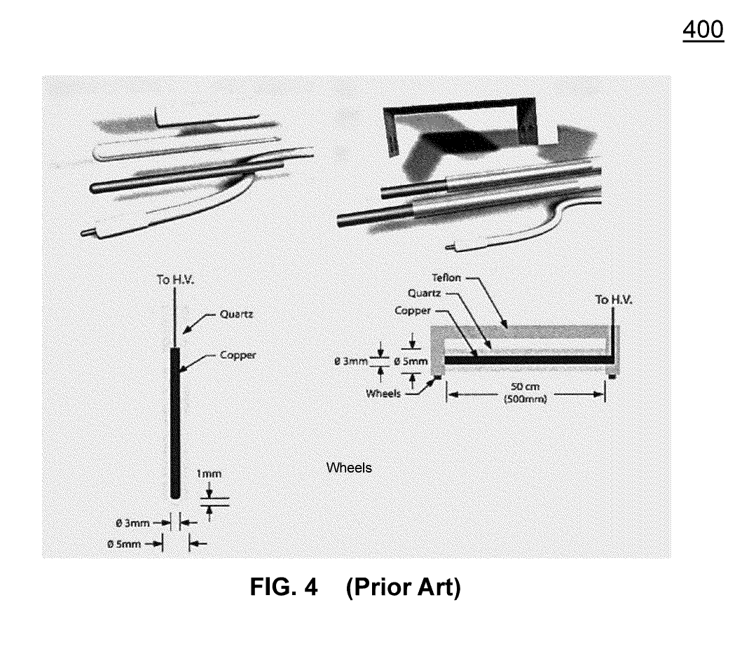

FIG. 5 illustrates a glass florescent light tube DBD wand device, in accordance with an embodiment of the present invention.

FIG. 6 illustrates a copper pipe, shrink wrapped DBD wand device, in accordance with an embodiment of the present invention.

FIG. 7 illustrates further details of a cold plasma wand device, in accordance with an embodiment of the present invention.

FIG. 8 illustrates a multi-element cold plasma DBD wand device with a plurality of electrodes, in accordance with an embodiment of the present invention.

FIG. 9 illustrates a fluorescent glass tube cold plasma DBD wand device, in accordance with an embodiment of the present invention.

FIG. 10 illustrates a sustained non-thermal plasma discharge in excess of 30 centimeters in length along the entire margin of the cylindrical electrode, in accordance with an embodiment of the present invention.

FIG. 11 illustrates the generation of plasma along the radius of the curve of the DBD wand device, in accordance with an embodiment of the present invention.

FIG. 12 illustrates a flowchart of a method that provides treatment distance control of a cold plasma device, in accordance with an embodiment of the present disclosure.

### DETAILED DESCRIPTION OF THE INVENTION

Cold temperature plasmas have attracted a great deal of enthusiasm and interest by virtue of their provision of plasmas at relatively low gas temperatures. The provision of plasmas at such a temperature is of interest to a variety of applications, including wound healing, anti-bacterial processes, treatments of musculoskeletal disorders, autoimmune disorder treatments and various other medical therapies and sterilization.

Embodiments of the present disclosure include cylindrical cold plasma DBD wand-like devices that provide a large cold plasma treatment area without the use of additional spatial control techniques. Powering these cylindrical cold plasma DBD devices with a multi-frequency harmonic-rich cold plasma (MFHCP) power supply avoids the formation of multiple discrete discharge points along the electrode (and associated pin-point heating and burning). The use of a MFHCP power supply results in a larger cold plasma treatment area (measured in centimeters or more) than that achievable (measured in millimeter values) with a single-frequency power supply. The dielectric of the DBD devices in embodiments of the present disclosure may be formed from polytetrafluoroethylene (PTFE), polyoxymethylene (POM), polyethylene (PE), polypropylene (PP), quartz, glass, or other dielectric materials known to one skilled in the art. The electrodes may be formed of a suitable metal or conductive gas separated from the target by the dielectric. Distribution of the energy can also be achieved by using a saline-filled DBD electrode, or a DBD electrode formed by metallic shavings (e.g., non-magnetic such as brass shavings) to improve the capacitance of the electrode and ensure cool discharge. The shapes of embodiments of the present disclosure are cylindrical, and may include a radiused tip. The larger variance in treatment distances available in embodiments of the present disclosure allows the inclusion of a handle for manual manipulation of the plasma. The use of a handle, or any form of manual manipulation, is not feasible with conventional DBD devices, as their plasma treatment distances must be precisely maintained and thereby be positioned with precision positioning equipment.

Embodiments described in the present disclosure can be directed to various medical treatment applications. In an exemplary embodiment, the cold plasma DBD wand device is powered by the multi-frequency harmonic-rich cold plasma (MFHCP) power supply (which generates a variety of harmonic frequencies simultaneously) and results in a cold plasma with a large treatment area (measured in centimeters or greater). As noted above, previous approaches would be unable to provide a large treatment area, as these prior approaches provided cold plasmas whose size was measured in millimeter values. The larger treatment areas available to embodiments described herein are useful for applications such as those described in U.S. application Ser. No. 14/026,679, entitled “Therapeutic Applications of Cold Plasma,” filed Sep. 13, 2013. The larger treatment areas available to embodiments described herein are also effective at treating various forms of musculoskeletal pain, fatigue, disorders and injuries. Musculoskeletal disorders (MSDs) can manifest in the upper or lower body. MSDs like fibromyalgia or work-related MSDs develop over time, affect the body's muscles, joints, tendons, ligaments, and nerves, and thereby greatly reduce a patient's quality of life. Due to the MFHCP devices' ability to reduce inflammation, deep tissue bruising can be cleared much more rapidly with the application of a MFHCP DBD device treatment protocol, thereby improving a patient's quality of life. In an embodiment, the MFHCP DBD wand device has been found to be effective in the treatment of tendonitis pain induced by repetitive stress. The MFHCP DBD wand device may also be effective at reducing spasticity in skeletal muscles caused by diseases affecting the central nervous system such as multiple sclerosis.

Still referring to embodiments of the present invention, the cold plasma DBD wand device's broad surface of plasma generation allows for the efficient treatment of larger areas with the benefits of being durable, portable, and being able to treat almost any anatomical structure. The terms “wand” and “wand device” are used to convey the notion that such a device is configured to deliver a cold plasma along a smooth peripheral area close to, and possibly including, its distal end, where the device is sufficiently small enough to negotiate placement at the desired treatment area without damaging either the treatment area, nearby regions or any regions encompassed during positioning of the wand device at the treatment area. Certain embodiments of the wand device may have a handle to enable negotiation of the wand device to the desired treatment area.

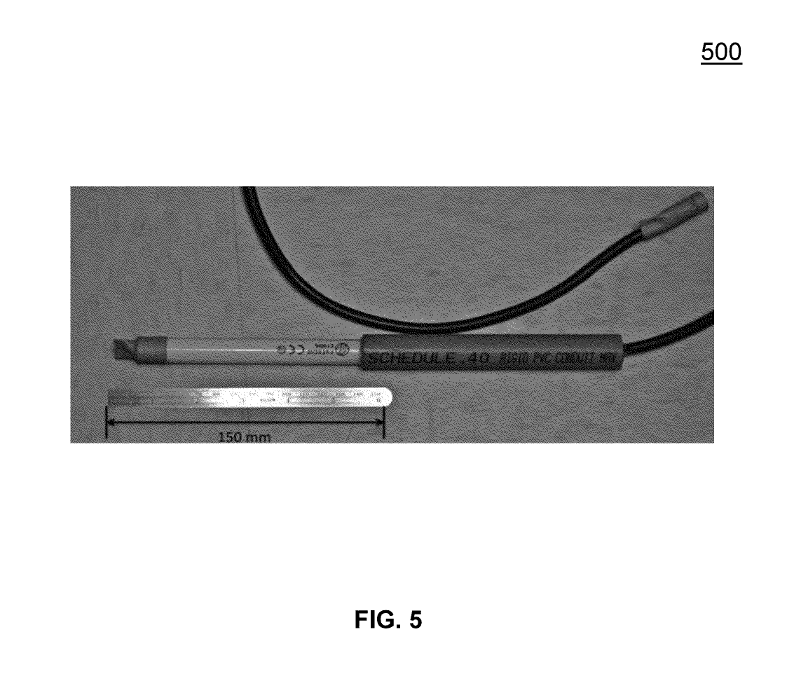

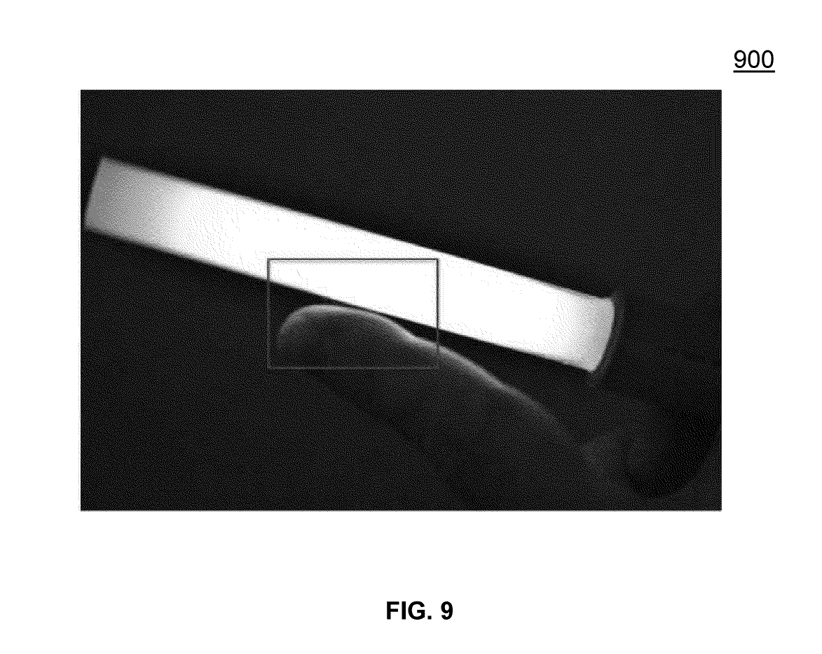

As noted above, embodiments of the present disclosure are cylindrical, receive high voltage internally, and have a dielectric barrier surrounding the inner, energized portion. In an embodiment, the MFHCP power source design (described in U.S. Provisional Patent Application No. 60/913,369, U.S. Non-provisional Application No. 12/038,159 (that has issued as U.S. Pat. No. 7,633,231) and the subsequent continuation applications (collectively “the '369 application family”), and the cold plasma high voltage power supply described in U.S. patent application Ser. No. 13/620,118 and U.S. Provisional Patent Application No. 61/535,250, which are incorporated herein by reference.) allows for a high level of ionization without substantial temperature rise due to its production of multiple frequencies. A further factor in the effective plasma delivery with a cold plasma DBD cylindrical device is the constant radius surface, which creates a tangential surface having an infinite number of discrete distances between the surface edge of the substrate under treatment and the wand device (see FIGS. 5 through 10), including excellent plasma generation along the margin between the wand device and the treatment surface (see FIGS. 9 through 12).

Further details of three of the embodiments of the invention are provided below.

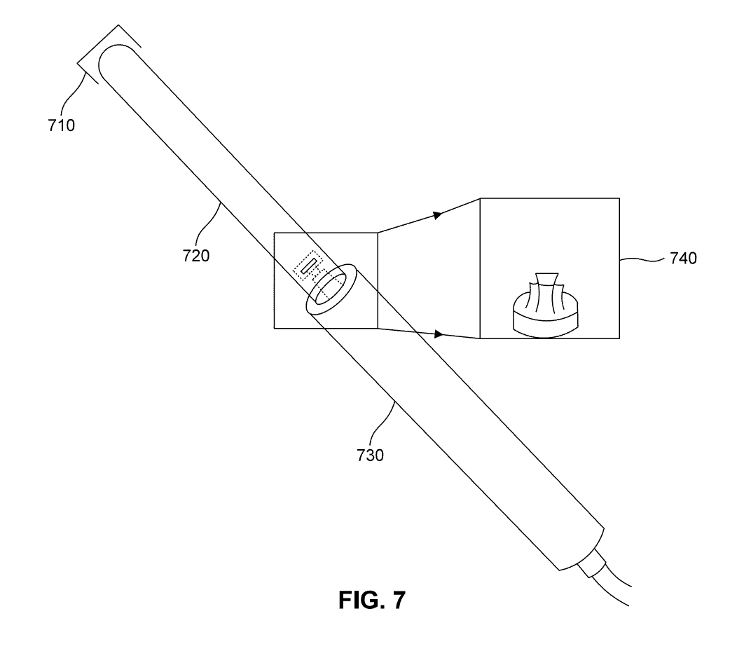

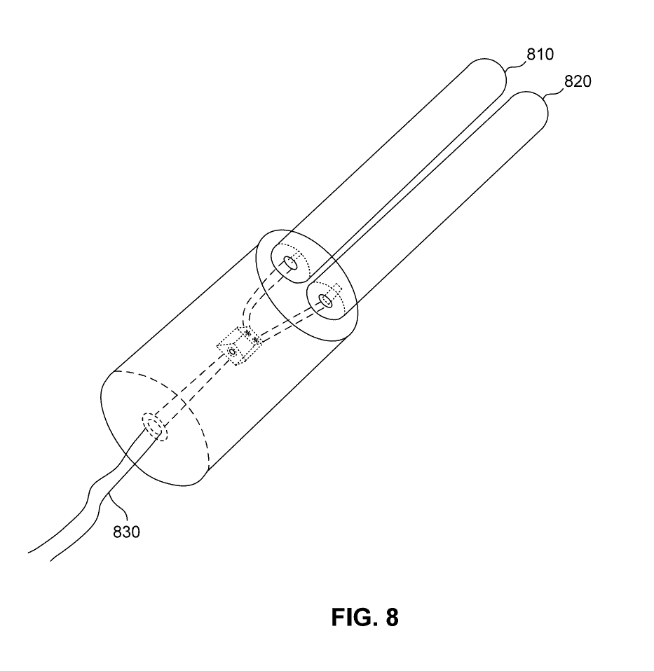

FIG. 5 illustrates one embodiment of the cold plasma DBD wand device. Referring to FIG. 5, the cold plasma DBD wand device may be fashioned out of a small florescent light bulb in which there is a low-pressure (e.g., <1 atm) gas (e.g., Hg) contained within the blub. The glass tube acts as a dielectric barrier in the DBD device, as described below. An end cap is placed over the terminals on the distal end of the fluorescent bulb to prevent uncontrolled discharge through these terminals. In an exemplary embodiment, a glass radius sealed tip may be used (see, e.g., FIGS. 7, 8). Such a glass radius sealed tip increases the available radii of curvature to address different sizes and shapes of body regions, thereby allowing the wand to be directed to very specific anatomical points as well as to be inserted into a bodily lumen. The proximal end of the bulb is placed and cemented into a corradial handle with insulative properties, in this example a length of schedule 40 rigid PVC conduit, for safe and effective hand-held operation of the unit. A high-voltage (HV) cable is used to connect the power supply to the fluorescent bulb DBD wand device through this handle. The MV cable is attached to one or both terminals on the proximal end of the fluorescent bulb.

The electrical energy is passed from the external pins into the inner portion of the fluorescent light bulb, with the glass functioning as a dielectric barrier, such that plasma is generated on the external surface of the bulb when brought into contract with a ground or floating ground. The fluorescent bulb feature of the glass DBD wand still functions as a light source since high voltage is being applied to the internal gas and therefore the device may be designed to emit specific wavelengths radiation to thereby allow for enhanced levels of treatment. For example, if an ultraviolet light is emitted along with the plasma, enhanced disinfection may be achieved when the goal of a plasma therapy is antibiotic/antiseptic in nature. The generated UV can also be used to effectively treat skin disorders such as psoriasis and vitiligo. This embodiment simultaneously generates and combines reactive oxygen species (ROS), reactive nitrogen species (RNS), charged particles, together with the electroporation effects of cold plasma with UV light. The simultaneous generation and combination within the same device and in close proximity to the target greatly enhances antisepsis, or treatment modalities, that can be achieved.

The cold plasma DBD wand device generates cold plasma wherever it comes into direct or proximate contact with the target (when sufficient ground potential exists). In an exemplary embodiment, optimal treatment distances between the wand device and the treatment target range from direct physical contact up to ˜2 mm, depending upon the voltage, frequency, substrate conductance, substrate capacitance, and the dielectric properties of the medium through which the plasma passes. It should be stressed that with a large diameter cylindrical electrode, such as the fluorescent light bulb, even when the surface is brought into direct contact with the substrate, there are discharge points along the entire radius of curvature at varying distances from the substrate. This is not the case with a planar electrode where the entire electrode is substantially the same distance from, or in direct contact with, the target substrate.

In a second embodiment, a standard ⅜ inch copper plumbing pipe is cut to size and a heavy-duty heat shrink wrap is added to the outer surface of the copper tubing to act as the dielectric barrier. Several advantages to the copper pipe DBD wand device are that it is less fragile, very inexpensive to manufacture, it cannot release toxic materials Hg) if broken, and any length and diameter can be selected. The “copper” based embodiments may be fanned in a number of different ways. An advantage of using a “copper pipe” embodiment over an alternative “solid copper rod” embodiment is a significant reduction in manufacturing costs and weight, particularly for large wand devices. Due to the use of MFHCP power source, MFHCP DBD wand devices are markedly larger in diameter and length than any of those previously developed, thereby creating a considerably more generous surface area for treatment.

Further details of embodiments of the present invention can be found by reference to the following figures.

FIG. 5 illustrates a UV generating glass florescent light tube DBD wand device, in accordance with an embodiment of the present invention.

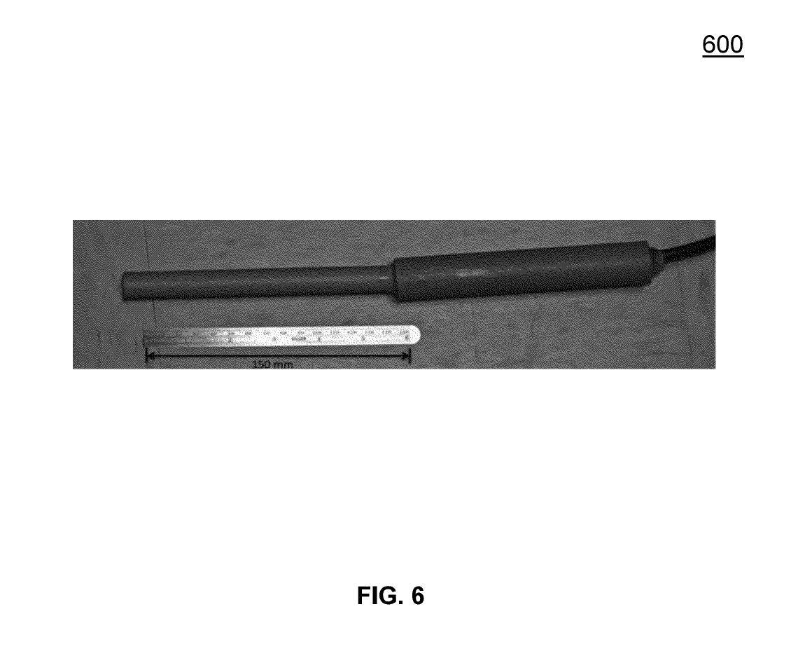

FIG. 6 illustrates a copper pipe, shrink wrapped DBD wand device, in accordance with an embodiment of the present invention.

FIG. 7 illustrates further details of a cold plasma wand device, in accordance with an embodiment of the present invention. In this embodiment, the radius tip of the cold plasma wand device allows for treatment at specific anatomical points and for insertion into bodily lumens. DBD wand device features a cold plasma DBD wand 720 together with handle 730. Cold plasma DBD wand 720 is typically rigid, and may be a glass tube. Cold plasma emanates from cold plasma DBD wand 720. Cold plasma DBD wand 720 has an electrode diameter that exceeds 1 cm, and can produce a cold plasma around its circumference along its length. Handle 730 is made of a suitable dielectric material, and is typically a rigid handle to permit the operator to manipulate the cold plasma DBD wand 720 so as to direct the resulting plasma to the treatment area of interest. Cold plasma DBD wand 720 may include a radius tip 710. Cold plasma emanates from the entire radius of radius tip 710, and therefore radius tip 710 is a functioning tip. Radius tip 710 is useful for certain treatments, as explained above. Supplying energy to cold plasma DBD wand 720 is bi-pin 740, which is coupled to high voltage RF input port 750 located typically at the end of handle 730. The general shape of the wand could include a constant radius of both the entire wand surface and the tip as pictured above, or a variable radius/curvature along the length and/or the distal tip.

FIG. 8 illustrates a multi-element cold plasma DBD wand device 800 with a plurality of electrodes, in accordance with an embodiment of the present invention. This embodiment has an element that is constructed out of copper and another element constructed out of a light-producing glass fluorescent tube for combined therapy applications. Element 810 provides a cold plasma, and may be formed from a copper electrode placed within a glass tube and coupled to high voltage RF input port 830. Element wand 820 provides a source of ultraviolet light, and may be formed from a fluorescent glass tube, with its element coupled to high voltage RF input port 830. The pictured embodiment highlights midpoint electrode placement of the copper electrode in the glass tube element, but the electrodes could be attached at the proximal end or at any desired point in the glass tubing. In terms of element 810, further embodiments could contain all copper, all glass, or a combination of electrodes in order to achieve the desired contact time and effect. Not shown in FIG. 8, a mirror may form part of multi-element cold plasma DBD wand device 800, where the mirror is placed behind element 820 element to direct ultraviolet let away from the operator and redirect the ultraviolet light toward the treatment area. The multi-element DBD wand can be powered by one multi-frequency harmonic-rich cold plasma (MFHCP) power supply (described in U.S. Provisional Patent Application No. 60/913,369, U.S. Non-provisional Application No. 12/038,159 (that has issued as U.S. Pat. No. 7,633,231) and the subsequent continuation applications (collectively “the '369 application family”), and the cold plasma high voltage power supply described in U.S. patent application Ser. No 13/620,118 and U.S. Provisional Patent Application No. 61/535,250, which are incorporated herein by reference.), or two or more such power units run in parallel.

FIG. 9 illustrates a fluorescent glass tube cold plasma DBD wand device, in accordance with an embodiment of the present invention. Note the plasma between the fingertip and the cold plasma. DBD wand device (boxed in red) coming off the radius edge of the device. In one embodiment, the glass DBD wand would generate selected wavelengths of light, such as UV, along with the plasma to produce a combined therapeutic effect. In another embodiment, the glass (or other dielectric) tube may be filled with a conductive solution (such as a saline solution) to form the DBD electrode. In a further embodiment, the glass (or other dielectric) tube may be filled with metallic shavings (e.g., non-magnetic such as brass shavings) together with a vacuum or appropriate gas to form the DBD electrode) inside the dielectric tube.

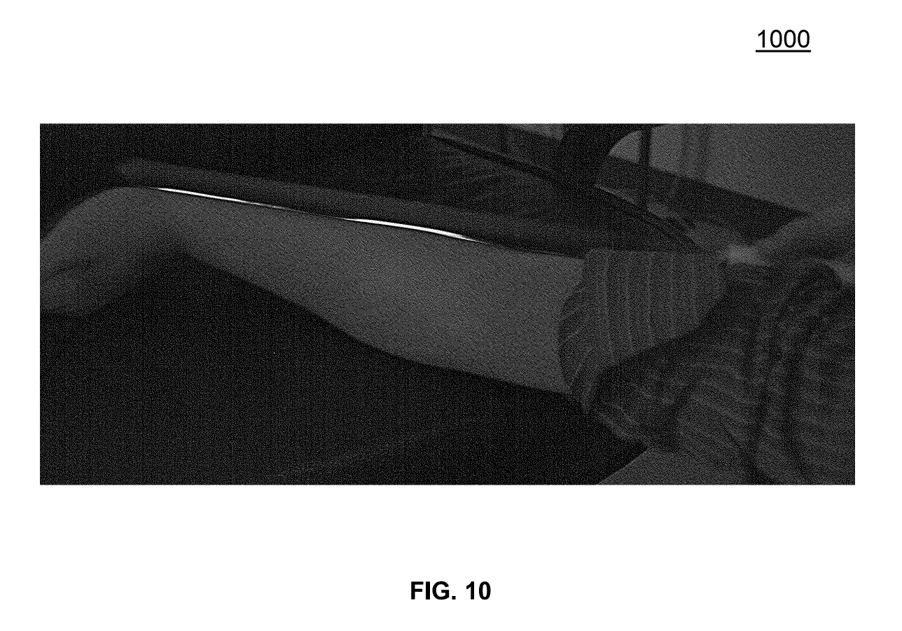

FIG. 10 illustrates a sustained non-thermal plasma discharge in excess of 30 centimeters in length along the entire margin of the cylindrical electrode, in accordance with an embodiment of the present invention. Note how the generated plasma originates from multiple distances around the radius of the curvature from the wand.

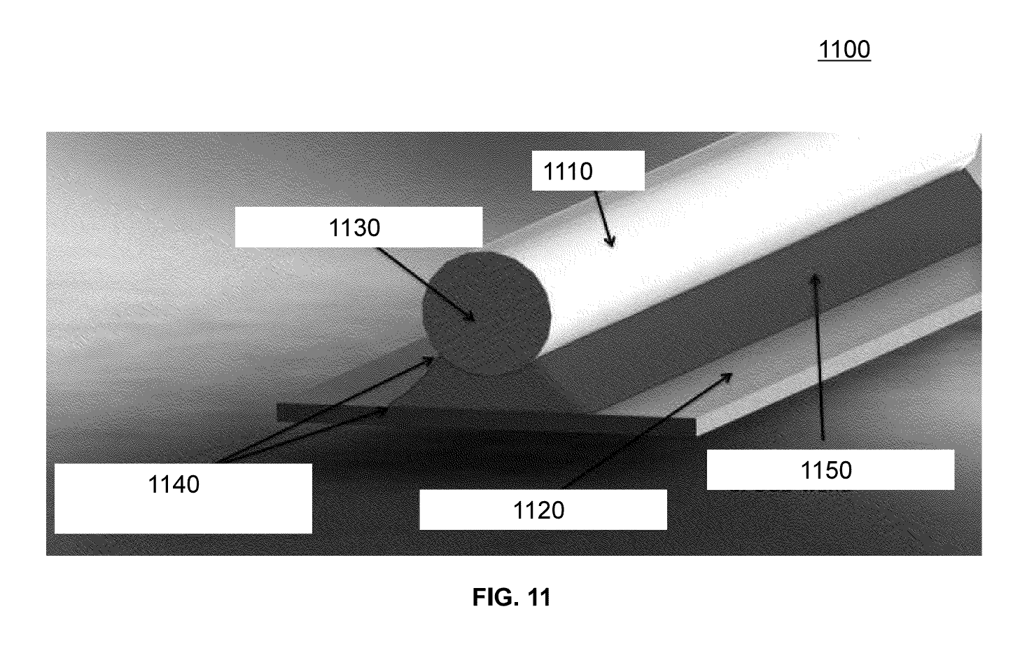

FIG. 11 illustrates the generation of plasma along the radius of the curve of the DBD wand device, in accordance with an embodiment of the present invention. FIG. 11 illustrates a cylindrical cold plasma DBD device 1110 applied to a flat substrate 1120 that is representative of a treatment area, in accordance with an embodiment of the present invention. Note the mantle of plasma 1150 generated along the margins of the electrode. Note also the plasma 1140 along the radius curve of electrode 1110 at its distal end 1130. Such a generation of plasma along the entire margin allows the plasma to cover large areas of the treatment substrate at one time.

The same core cold plasma DBD device may be employed with a carrier in an industrial process setting. For example, the same core cold plasma DBD device may be employed in a food processing setting, as further explained in U.S. patent application Ser. No. 14/103,540, filed Dec. 11, 2013, which is incorporated herein by reference in its entirety. When the core cold plasma DBD device is used in a setting where manual manipulation is used (e.g., clinician setting), a handle is typically attached. Thus, from a terminology point of view, the term “wand” is used herein to denote the attachment of a handle such that manual manipulation of the cold plasma device may be accomplished.

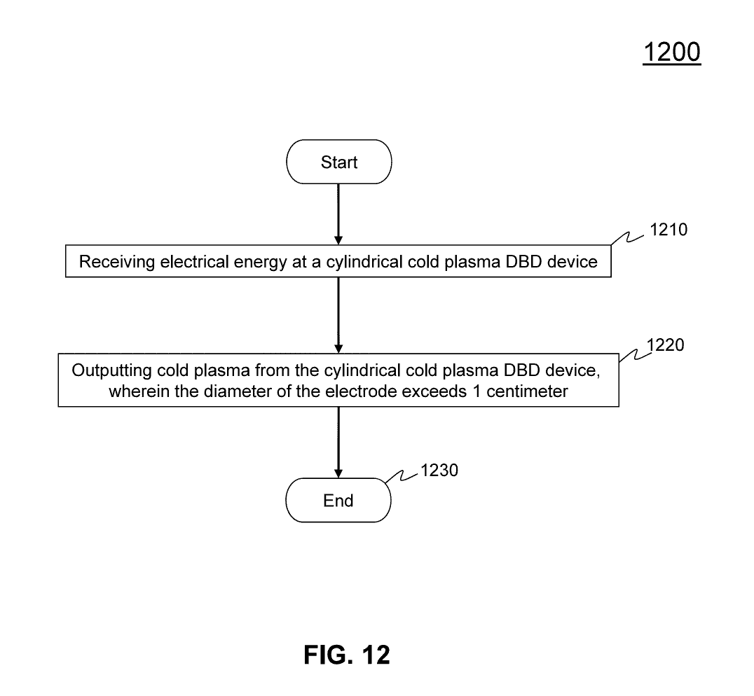

FIG. 12 provides a flowchart of a method that provides for the outputting of cold plasma, according to an embodiment of the current invention.

The process begins at step 1210. In step 1210, electrical energy is received at a cylindrical cold plasma DBD device. In an embodiment, cylindrical cold plasma device 800 receives the electrical energy.

In step 1220, cold plasma is output at a target substrate from the cylindrical cold plasma DBD device, wherein the diameter of the electrode is in excess of 1 centimeter.

At step 1230, method 1200 ends.

The above disclosure provides various embodiments of cylindrical cold plasma

DBD devices that provide a large cold plasma treatment area. In the context of this application, a large cold plasma treatment area refers to the need to project a cold plasma to a target treatment area using a cold plasma DBD electrode having a diameter in excess of 1 centimeter, something that has not been achievable by prior approaches. The ability to project over such distances by cylindrical cold plasma DBD devices is enabled by the use of a multi-frequency harmonic-rich cold plasma (MFHCP) power supply. Such a supply avoids the formation of multiple discrete discharge points along the electrode (and associated pin-point heating and burning). Consequently, the use of a MFHCP power supply results in a larger cold plasma treatment area (measured in centimeters or more) than that achievable with a single-frequency power supply. Distribution of the cold plasma energy can also be achieved by using a saline-filled DBD electrode, or by using a DBD electrode formed by metallic shavings (e.g., non-magnetic such as brass shavings). The dielectric of the DBD devices in embodiments of the present disclosure may be formed from polytetrafluoroethylene (PTFE), polyoxymethylene (POM), polyethylene (PE), polypropylene (PP), quartz, glass, or other dielectric materials known to one skilled in the art. The shapes of embodiments of the present disclosure are cylindrical, and may include a radiused tip.

As discussed above, the larger electrode diameters available in embodiments of the present disclosure allows for the inclusion of a handle for manual manipulation of the plasma. The use of a handle, or any form of manual manipulation, is not feasible with conventional DBD devices, as they must be positioned with precision positioning equipment. Thus, the cylindrical cold plasma DBD device may be passed over the treatment area or substrate, or the cylindrical cold plasma DBD device may be stationary with the substrate moving in proximity to the cylindrical cold plasma DBD device.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. An apparatus comprising:
a cylindrical cold plasma dielectric barrier discharge (DBD) device coupled to a power supply, the cold plasma DBD device configured to direct a cold plasma at a target substrate, wherein an electrode of the cold plasma DBD device has a diameter in excess of 1 centimeter.

2. The apparatus of claim 1, further comprising:
a handle mechanically coupled to the cold plasma DBD device, the handle having insulative properties.

3. The apparatus of claim 1, wherein the cylindrical cold plasma DBD device includes a radius tip at an end of the cylindrical cold plasma DBD device.

4. The apparatus of claim 1, wherein the cylindrical cold plasma DBD device includes a radiused surface that, when placed on a flat surface of the target substrate, provides tangential contact that leads to multiple numbers of distances between a flat surface of the target substrate under treatment and the cylindrical cold plasma DBD device.

5. The apparatus of claim 1, wherein the cold plasma DBD device includes a fluorescent tube having the cylindrical shape.

6. The apparatus of claim 5, wherein the fluorescent tube includes an internal electrode coupled to the power supply, the internal electrode having a bi-pin element.

7. The apparatus of claim 1, wherein the cold plasma DBD device includes a copper cylinder having an insulator cladding around the copper cylinder.

8. The apparatus of claim 1, further including a second cold plasma DBD device having a cylindrical shape that, when placed on a flat surface of the target substrate, provides tangential contact that leads to multiple numbers of distances between a flat surface of the target substrate under treatment and the second cold plasma DBD device.

9. The apparatus of claim 1, further including a reflecting mirror configured to redirect UV emissions associated with the cold plasma towards the target substrate.

10. The apparatus of claim 1, wherein the cylindrical cold plasma DBD device includes an internal electrode that includes a tube filled with a saline solution.

11. The apparatus of claim 1, wherein the cylindrical cold plasma DBD device includes an internal electrode that includes a glass tube filled with non-magnetic metallic shavings and a noble gas.

12. The apparatus of claim 1, further comprising:
a cold plasma power supply coupled to the cold plasma device, the cold plasma power supply configured to provide the cylindrical cold plasma device with an electrical voltage having two or more harmonic frequencies.

13. A method comprising:
receiving, from a power supply, electrical energy at a cylindrical cold plasma dielectric barrier discharge (DBD) device; and
outputting the cold plasma at a target substrate, wherein an electrode of the cold plasma DBD device has a diameter in excess of 1 centimeter.

14. The method of claim 13, wherein the cylindrical cold plasma DBD device includes a radius tip at an end of the cylindrical cold plasma DBD device.

15. The method of claim 13, wherein the cylindrical cold plasma DBD device includes a radiused surface that, when placed on a flat surface of the target substrate, provides tangential contact that leads to multiple numbers of distances between a flat surface of the target substrate under treatment and the cylindrical cold plasma DBD device.

16. The method of claim 13, wherein the cold plasma DBD device includes a fluorescent tube having the cylindrical shape.

17. The method of claim 13, wherein the cold plasma DBD device includes a copper cylinder having an insulator cladding around the copper cylinder.

18. The method of claim 13, further comprising:
receiving, from a power supply, electrical energy at a second cold plasma dielectric barrier discharge (DBD) device, the second cold plasma DBD device having a wand-like shape to create a tangential surface with a large number of distances between a surface of a target substrate under treatment and the cold plasma DBD device; and
outputting the cold plasma at the target substrate over an effective area.

19. The method of claim 13, further comprising:
using a reflecting mirror configured to redirect UV emissions associated with the cold plasma towards the target substrate.

20. The method of claim 13, wherein the cold plasma device includes an array of DBD wand devices.

21. The method of claim 13, wherein the cold plasma device further includes an internal electrode coupled to the power supply, the internal electrode having a bi-pin element.

22. The method of claim 13, wherein the cylindrical cold plasma DBD device includes an internal electrode that includes a tube filled with a saline solution.

23. The method of claim 13, wherein the cylindrical cold plasma DBD device includes an internal electrode that includes a glass tube filled with non-magnetic metallic shavings and a noble gas.

24. The method of claim 13, wherein the outputting the cold plasma includes:
using a handle mechanically coupled to the cylindrical cold plasma DBD device, the handle having insulative properties.

25. The method of claim 13, wherein the receiving electrical energy includes:
receiving energy from the cold plasma power supply, wherein the cold plasma power supply is configured to provide the cylindrical cold plasma DBD device with an electrical voltage having a multi-frequency harmonic-rich content.

26. An apparatus comprising:
an array of multi-frequency harmonic-rich (MFHCP) powered cylindrical dielectric barrier discharge (DBD) devices coupled to one or more MFHCP power supplies, the array of cylindrical cold plasma DBD devices each having a radiused surface that, when placed on a flat surface of the target substrate, provides tangential contact that leads to multiple numbers of distances between a flat surface of the target substrate under treatment, and configured to direct a cold plasma at the target substrate.
